(19) United States
(12) Patent Application Publication
(10) Pub. No.: US 2009/0076853 A1
(43) Pub. Date: Mar. 19, 2009

(54) SYSTEM, APPARATUS AND METHOD OF MAKING IMAGE INTERPRETATION REPORT

(75) Inventor: Masafumi Sagawa, Minato-ku (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM CORPORATION, Tokyo (JP)

(21) Appl. No.: 12/208,045

(22) Filed: Sep. 10, 2008

(30) Foreign Application Priority Data
Sep. 14, 2007 (JP) 2007-238813

(51) Int. Cl. A61B 5/00

(52) U.S. Cl. 705/3

(57) ABSTRACT

To make an image interpretation report on present examination images like CT slice images taken from a patient, a report server is searched for a past report on past examination images taken from the same patient under similar condition. The past report contains an original image link data for linking a key image to an original image that is one of the past examination images. Concerning imaging positions, correspondence data representative of correspondence between the present images and the past images is produced. With reference to the original image link data of the past report and the correspondence data, a present key image is produced from one of the present images, which corresponds in imaging position to the original image of the past key image. Based on data relating to the present key image and data contained in the past report, a template for the present report is displayed on a screen.

### FIELD OF THE INVENTION

The present invention relates to an image interpretation report making system for making an update report on an examination for follow-up observation of a patient on the basis of past image interpretation reports of the same patient. The present invention relates also to an image interpretation report making apparatus and an image interpretation report making method.

### BACKGROUND OF THE INVENTION

In medical facilities like hospitals, a variety of medical examination apparatuses, such as modalities for CR (computed radiography), CT (computer tomography), MRI (magnetic resonation imaging), PET (positron emission tomography) and ultrasonic diagnosis, have been widely used. Medical images taken by the modalities are used in medical diagnoses, taking an important roll on determining the nature of a condition of a patient.

For the diagnosis based on medical images, doctors of individual diagnosis and treatment departments generally ask a doctor in an inspection department, like a radiologist, for an interpretation of the medical images. In the inspection department, the doctor interprets the medical images and reports the interpretation. Then, the doctor in the diagnosis and treatment department examines the report on the image interpretation, hereinafter referred to as the image interpretation report, to diagnose a disorder (an injury or a disease) of the patient.

Recently, in order to improve work efficiency in the medical field, computerization of medical images and charts has been promoted. Under this trend, systems for making the above-mentioned image interpretation online have been suggested for example in JPA2007-122679.

In practice, medical examinations are often carried out periodically on the same patient at an interval of three months, six months or one year, to observe the course of an illness or the like. Concerning such examinations for follow-up observation, the doctor will make the image interpretation while monitoring past images obtained through past inspections or examinations as well as present images obtained through the latest inspection or examination, so the doctor may compare the present images with the past images to find out any differences between them, for example a change in size of an affected part. The result of comparison is written as a finding in the report.

According to the image interpretation support system of the above-mentioned prior art, comments written in an image interpretation report on the past examination, such as findings, opinions and diagnoses, are copied in an image interpretation report on the present examination, for use as a draft of comments on the present examination. So the doctor in the inspection department may make the present image interpretation report on the present examination just by rewriting the draft. Thus, this prior art image interpretation support system facilitates creation of the image interpretation reports concerning the follow-up observation examination, and thereby reduces labor of the doctors in the inspection department.

Besides the comments, the image interpretation report contains information on medical images, such as a key image that attracted doctor's attention among the examination images during the image interpretation or link information for reading out automatically such an image that the doctor considers to pay attention to. For the sake of reducing doctor's labor in the inspection department, it is desirable that information on the past medical images is automatically input in the present image interpretation report. The prior art image interpretation support system, however, does not consider the information on medical images at all. That is, in the prior art system, doctors have to select a key image from among a series of examination images, and copy and paste the key image in the image interpretation report or correlate it with the link information all by themselves, even while they are making reports on a follow-up observation examination. Therefore, the prior image interpretation support system does not sufficiently facilitate making the image interpretation reports.

Particularly on those examinations using CT or MRI scanners, tens or hundreds of images are taken at each examination. It has been labor-consuming for the doctors to retrieve such an image from among a large number of newly taken examination images that corresponds in location of the imaged subject to a key image of the past examination of the same case. Therefore, a system has been desired that makes it unnecessary for the doctors to copy and past the key image or correlate it with the link information and thus facilitates making the image interpretation report.

As a method of inputting information on medical images automatically in the present image interpretation report, it may be possible to produce a key image and link information from the present images. But where there are many past images and present images, it is hard to determine correspondence between the respective imaging positions of the present images and those of the past images. It may also be possible to make a copy of information on medical images from the past image interpretation report. But a simple copy of the past information has a problem that the past examination images are linked to the present image interpretation report.

### SUMMARY OF THE INVENTION

In view of the foregoing, a primary object of the present invention is to provide a system, an apparatus and a method, which make it possible to make an image interpretation report on a follow-up observation examination without the need for inputting information on medical images.

An image interpretation report making system of the present invention comprises a correspondence data producing device for producing correspondence data that shows correspondence between present images and past images with respect to their imaging positions; and a template producing device for producing a template for a present report on the basis of the correspondence data and a past report.

The past report depicts an interpretation of the past images, whereas the present report depicts an interpretation of the present images. The past images were taken from a test device at diverse imaging positions and are stored in a data storage device as part of past data obtained through a past examination done on the test device, whereas the present images were taken from the same test device at similar imaging positions to the past images through an examination lately done on the same test device under similar condition to the past examination. The past report and the present report are stored in a report storage device.

The template may be displayed on a display device, and is edited on the display device, to complete the present report.

Preferably, the template producing device refers to the correspondence data to produce information relating to the present images from information relating to the past images that is included in the past data, and produces the template on the basis of the information relating to the present images.

The past report preferably includes a key image as selected from the past images, and the information relating to the past images includes past key image data that shows the key image of the past report, and wherein the template producing device produces a present key image and present key image data from one of the present images that is retrieved from the data storage device with reference to the correspondence data and the past key image data, and includes the present key image data in the information relating to the present images.

Preferably, the information relating to the past images includes annotation data that represents an annotation consisting of text data and a mark that is put on the past key image, and the template producing device copies the annotation data and includes it as annotation data for the present key image in the information relating to the present images.

More preferably, the information relating to the past images includes link data that links the annotation to comments written in the past report, and the template producing device copies the link data to include it in the information relating to the present images.

The comments preferably include a finding that describes a condition seen from the past images and/or a diagnosis that describes a symptom or an impression derived from the finding.

An image interpretation report making method for making a present report on the basis of a past report comprises steps of producing correspondence data that shows correspondence between present images and past images with respect to their imaging positions; and producing a template for a present report on the basis of the correspondence data and the past report, wherein the past report depicts an interpretation of the past images taken from a test device at diverse imaging positions at a past examination, whereas the present report depicts an interpretation of the present images lately taken from the same test device under similar condition to the past examination.

According to the present invention, the template producing device automatically produces the template for the present report on the basis of the past report and the correspondence data that shows the correspondence between the present images and the past images with respect to their imaging positions. According to the preferred embodiment, the template for the present report is produced by producing the information relating to the present images from the information relating to the past images, as well as by copying the comments as written in the past report into the template. Therefore, the doctor in charge of making the image interpretation report can make the present report with ease just by editing the template.

### BRIEF DESCRIPTION OF THE DRAWINGS

The above and other objects and advantages of the present invention will be more apparent from the following detailed description of the preferred embodiments when read in connection with the accompanied drawings, wherein like reference numerals designate like or corresponding parts throughout the several findings, and wherein:

FIG. 1 is an explanatory diagram schematically illustrating a medical network system;

FIG. 2 is an explanatory diagram schematically illustrating of a structure of volume data and slice images;

FIG. 3 is a block diagram schematically illustrating a structure of an image interpretation report making terminal;

FIG. 4 is a block diagram schematically illustrating a structure of an image server;

FIG. 5 is an explanatory diagram illustrating a concept of correspondence between present data and past data;

FIG. 6 is an explanatory diagram illustrating an example of correspondence data;

FIG. 7 is an explanatory diagram illustrating an example of an image display screen;

FIG. 8 is an explanatory diagram illustrating an example of a report creation screen;

FIG. 9 is an explanatory diagram illustrating a condition of the report creation screen, wherein a finding is highlighted;

FIG. 10 is an explanatory diagram schematically illustrating a structure of an image interpretation report;

FIG. 11 is an explanatory diagram illustrating an example of a mode selection screen;

FIG. 12 is an explanatory diagram illustrating a condition of the mode selection screen, wherein a warning window is overlaid;

FIG. 13 is an explanatory diagram illustrating a concept of copying respective data from a past report in a present report; and

FIG. 14 is a flowchart illustrating a sequence of procedures for making the image interpretation report.

### DESCRIPTION OF THE PREFERRED EMBODIMENTS

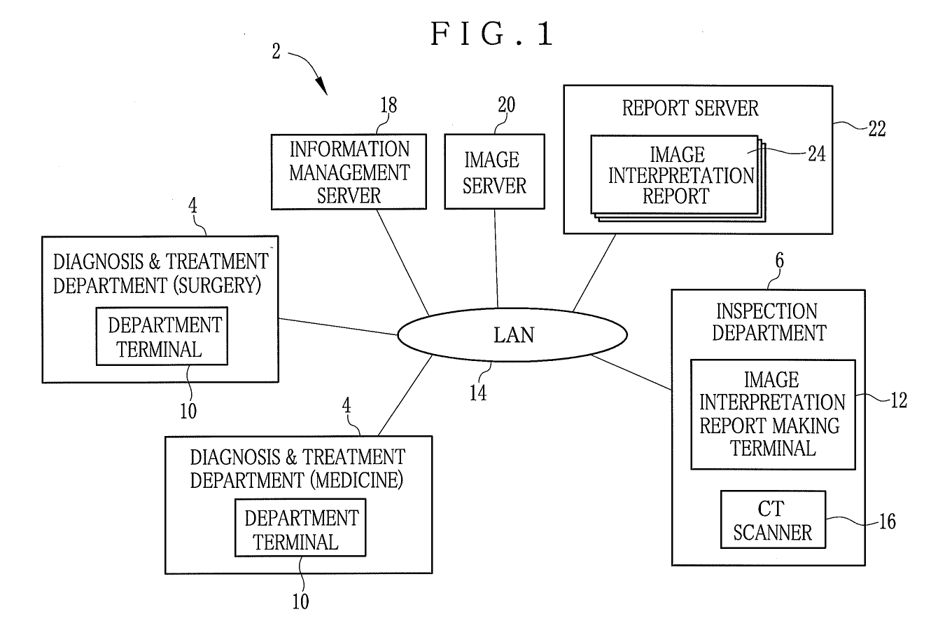

FIG. 1 shows a medical network system 2, which is installed in a medical facility like a hospital, as an embodiment of the image interpretation report making system of the present invention. The medical network system 2 consists of a plurality of department terminals 10 that are installed in respective diagnosis and treatment departments 4 like those for surgery, medicine etc., an image interpretation report making terminal 12 that is installed in an inspection department 6 like that for radiography, and a local area network (LAN) 14 interconnecting these terminals 10 and 12. To the LAN 14 are also connected a CT scanner 16, which is installed in the inspection department 6, an information management server 18, an image server 20 and a report server 22. The information management server 18 manages a variety of information inside the medical facility. The image server 20 is a data storage device storing various medical images including slice images 30 taken by the CT scanner 16. The report server 22 is a report storage device storing image interpretation reports 24, in which findings, opinions and diagnosis obtained by the image interpretation are written as comments.

A doctor in the individual diagnosis and treatment department 4 asks the inspection department 6 for an examination if it is necessary for diagnosing a disorder of a patient. In the inspection department 6, slice images 30 are taken from the patient according to the request from the diagnosis and treatment department 4. A doctor in the inspection department 6 interprets the slice images 30, makes an image interpretation report 24 on the results of interpretation of the slice images 30, and forwards the image interpretation report 24 back to the doctor who ordered the examination. On the basis of the image interpretation report 24, the doctor in the diagnosis and treatment department 4 finally makes a diagnosis. The medical network system 2 connects the respective department terminals 10 to the image interpretation report making terminal 12 via the LAN 14, so that the requests for examinations and the reports on image interpretation may be exchanged online, improving work efficiency in the medical facility. Note that more than one image interpretation report making terminal 12, more than one information management server 18, more than one image server 20 or more than one report server 22 may be connected to the LAN 14.

The information management server 18 is, for example, an HIS (hospital information system) server or a RIS (radiology information system) server. The information management server 18 manages various information including patient information, diagnosis and treatment information, examination information and account information in relation to respective patients. The patient information represents personal information on the individual patient, for example, patient's name, ID, address, birthday, age, sex, family, an amnesis or past illnesses and allergies.

The diagnosis and treatment information represents information on the diagnosis and treatment made to the patient, including the date of medical session, the department in charge, the name of disorder, the result of diagnosis, the duration of therapy, the kinds and amounts of prescribed drugs, the name of pharmacy in charge of medication, etc. The duration of therapy represents how long the patient has come to the medical facility for the therapy of the same disorder. The examination information represents information on medical images taken at each examination, including the date of examination, the machine used for the examination, the method of the examination and the site under the examination, etc. The information on the method of the examination includes directions and angles of taking the respective medical images, such as front and side, as well as whether radiopaque is used or not. The examined sites means body sites to be examined, such as head, neck, chest region, abdominal region, pelvic region, leg, trunk of body, etc. The account information includes for example charges for medical sessions, medications and inspections as well as whether the coverage is available or not.

The image server 20 is a so-called PACS (picture archiving and communication system for medical application) server. Beside the slice images 30, the image server 20 stores medical images sent from other medical facilities via a network or media and referential images for comparison between symptoms. The medical images are individually read out from the image server 20 to the department terminal 10 or the image interpretation report making terminal 12 according to the needs, so that doctors use them for image interpretation or explanation to the patient. Note that the medical images include not only the slice images 30 taken by the CT scanner 16 but also images taken by other modalities like CR machines and MRI scanners. The referential images include images showing various cases, which are taken by the modalities, and anatomic illustrations as well.

The report sever 22 stores the image interpretation reports 24 that are made as electronic data on the image interpretation report making terminal 12, and transfers the image interpretation reports 24 to the respective terminals 10 according to the demands from these terminals 10.

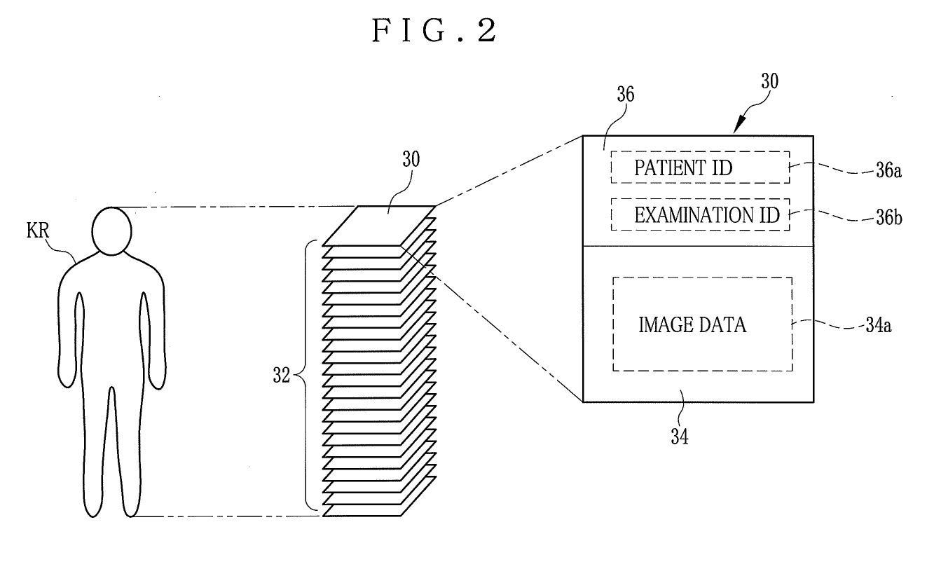

The CT scanner 16 takes a designed number of slice images 30 from a patient KR, as shown in FIG. 2, according to the order designating the slicing thickness and other set values for each individual examination. The slice images 30 obtained for each examination are transferred to the image server 20 and stored there in a group for each examination. Hereinafter, the group of slice images 30 will be referred to as volume data 32. If the volume data 32 contain images taken from more than one site, the images may be taken in continuous succession or with intermissions between different sites.

As shown in FIG. 2, the slice image 30 individually has an image recording area 34 for recording image data 34a and a tag area 36 for recording metadata, such as patient ID 36a, examination ID 36b and the like. The patient ID 36a is used to identify the patient KR whom the slice image 30 was taken from. The examination ID 36b is a specific number given to each examination, so that it is possible to identify the examination that the slice image 30 was taken for. With these IDs 36a and 36b, the slice images 30 for the same examination may be managed as a set of volume data 32.

Note that the metadata is not limited to the above embodiment, but any other information is recordable in the tag area 36 insofar as the information can identify the individual slice image 30. As an example of file format for such a medical image as having the tag area 36, DICOM (digital imaging and communications in medicine) format is usable.

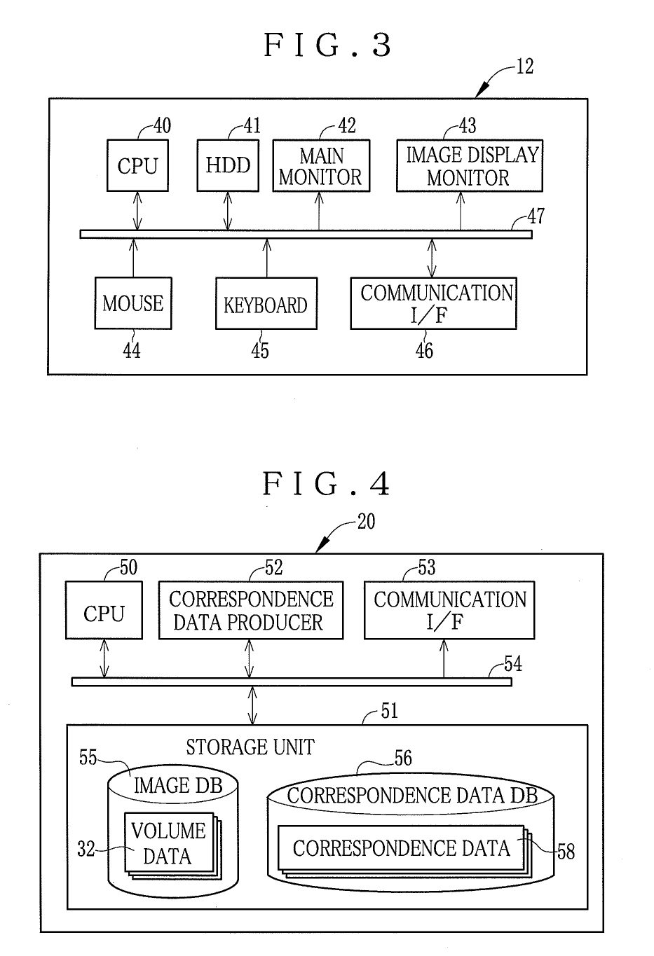

FIG. 3 schematically shows an internal structure of the image interpretation report making terminal 12. The image interpretation report making terminal 12 may be a well-known personal computer or work station. The image interpretation report making terminal 12 is provided with a CPU 40 supervising respective components of the image interpretation report making terminal 12, an HDD 41 that stores various programs related to the medical network system 2, a main monitor 42 for displaying various work screens, an image display monitor 43 for displaying the slice images 30, well-known input devices like a mouse 44 and a keyboard 45, and a communication interface (I/F) 46 for connecting the image interpretation report making terminal 12 to the LAN 14. These components are interconnected via a bus 47. The CPU 40 sequentially processes the program as read from the HDD 41, to control the image interpretation report making terminal 12.

The image interpretation report making terminal 12 has a so-called dual monitor structure as having the monitors 42 and 43. The image display monitor 43 is a high-definition monitor for displaying the slice images 30 clearly. While observing to interpret the slice images 30 displayed on the image display monitor 43, the doctor in charge of the image interpretation makes the image interpretation report 24 on a screen displayed on the main monitor 42. With these two monitors 42 and 43, the doctor can work out the image interpretation and the report making simultaneously on different screens, so the work efficiency of the image interpretation in the inspection department 6 is improved. However, the image interpretation report making terminal 12 is not limited to the dual monitor structure, but may be provided with a single monitor or more than two monitors.

FIG. 4 schematically shows an internal structure of the image server 20, which is provided with a CPU 50 supervising respective components of the image server 20, a storage unit 51 storing various programs related to the medical network system 2, a correspondence data producer 52 for producing correspondence data 58, and a communication interface (I/F) 53 for connecting the image server 20 to the LAN 14. These components are interconnected via a bus 54. The CPU 50 reads the program from the storage unit 51 and sequentially processes the program to control the image server 20.

The storage unit 51 also constitutes an image database (DB) 55 storing various medical images including the volume data 32, and a corresponding information database 56 storing the correspondence data 58 as produced by the correspondence data producer 52. The storage unit 51 is for example a disc array that consists of a series of hard disc drives. Note that the storage unit 51 does not necessarily store both the programs and the image data, but it is possible to provide separate storage units.

In medical facilities like hospitals, medical examinations are often carried out periodically on the same patient at an interval of three months, six months or one year, to observe the course of an illness or the like. For an follow-up examination, the inspection department 6 takes the slice images 30 under the approximately same conditions as in the previous examination, so that the taken slice images 30 may be compared with those taken in the previous examination, wherein the conditions include positions of the site for observation and display conditions of the images.

The correspondence data producer 52 creates the correspondence data 58 that represents correspondence between the slice images 30 contained in the volume data 32 as obtained at the previous examination and the slice images 30 contained in the volume data 32 as obtained newly at the follow-up examination.

As the volume data 32 is newly sent from the CT scanner 16, the correspondence data producer 52 refers to the respective tag areas 36 of the slice images 30 contained in the newly sent volume data 32, to check the patient ID 36a and the examination ID 36b. On the basis of the confirmed IDs 36a and ID 36b, the correspondence data producer 52 reads out examination information of that volume data 32 from the information management server 18.

On the basis of the examination information read out from the volume data 32, the correspondence data producer 52 checks if the examination was carried out under the similar conditions as in the past examination of the same patient. Thereby, the correspondence data producer 52 judges whether the volume data 32 as sent from the CT scanner 16 was obtained at an initial examination of a case, or at a follow-up examination. If the correspondence data producer 52 judges the volume data 32 to be obtained at the initial examination, it stores the volume data 32 in the image database 55.

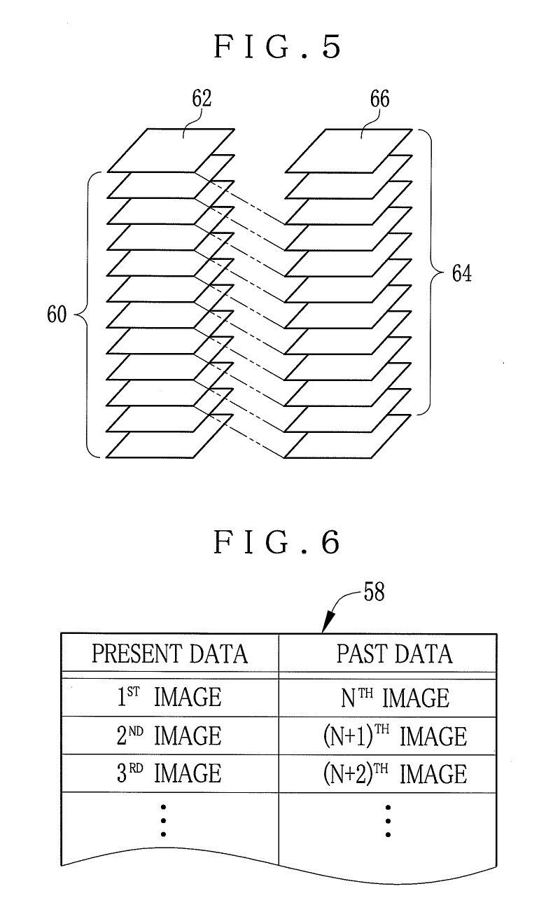

On the other hand, if the volume data 32 as sent from the CT scanner 16 is judged to be obtained at the follow-up examination, the correspondence data producer 52 reads out the volume data 32 as obtained at the previous examination, hereinafter referred to as the previous volume data 32, from the image database 55 on the basis of search results of the information management server 18. After reading the previous volume data 32, the correspondence data producer 52 determines the imaging position of each individual slice image 30 of the previous volume data 32 as obtained at the follow-up examination, to discriminate which slice images of the previous volume data 32 correspond to the respective slice images of the present volume data 32 with respect to their imaging positions. As shown in FIG. 5, the volume data 32 obtained newly at a follow-up examination will be referred to as present data 60, and slice images 30 contained in the present data 60 will be referred to as present images 62. On the other hand, the volume data 32 obtained at the previous examination will be referred to as past data 64, and slice images 30 contained in the past data 64 will be referred to as past images 66.

The judgment as to which past images 66 correspond to the respective present images 62 with respect to their imaging positions is made by calculating the respective imaging positions of the individual images 62 and 66 on the basis of some factors contained in the examination information, such as an initial imaging position, a final imaging position and a slicing thickness. The correspondence data producer 52 judges the correspondence of the individual present images 62 to the past images 66, and provides the result as the correspondence data 58. Note that the method of finding the correspondence between the present images 62 and the past images 66 is not limited to the above embodiment, but another method such as image analysis may be applicable.

The correspondence data 58 is produced for example in the form of a table that shows the respective serial numbers of those past images 66 which correspond to the present images 62 with regard to their imaging positions, as shown in FIG. 6. After producing the correspondence data 58, the correspondence data producer 52 writes the correspondence data 58 in the correspondence data database 56 and the present data 60 as well as the past data 64 in the image database 55. At that time, the correspondence data 58 is associated with the present data 60, for example by attaching the patient ID and the examination ID as metadata to the correspondence data 58.

Thus, the correspondence data producer 52 correlates the present images 62 with the past images 66 through the correspondence data 58. Note that the method of correlating the present images 62 with the past images 66 is described in more detail for example in JPA 2007-066016. Although the correspondence data 58 is produced as a data table in the present embodiment, the correspondence data 58 is not limited to this embodiment, but may be produced in association with each individual present image 62, for example, by recording information on the corresponding past image 66 as metadata in the tag area 36 of each present image 62. It is possible that the doctor in charge of image interpretation can manually produce and edit the correspondence data 58 by correlating the respective present images 62 with the past images 66.

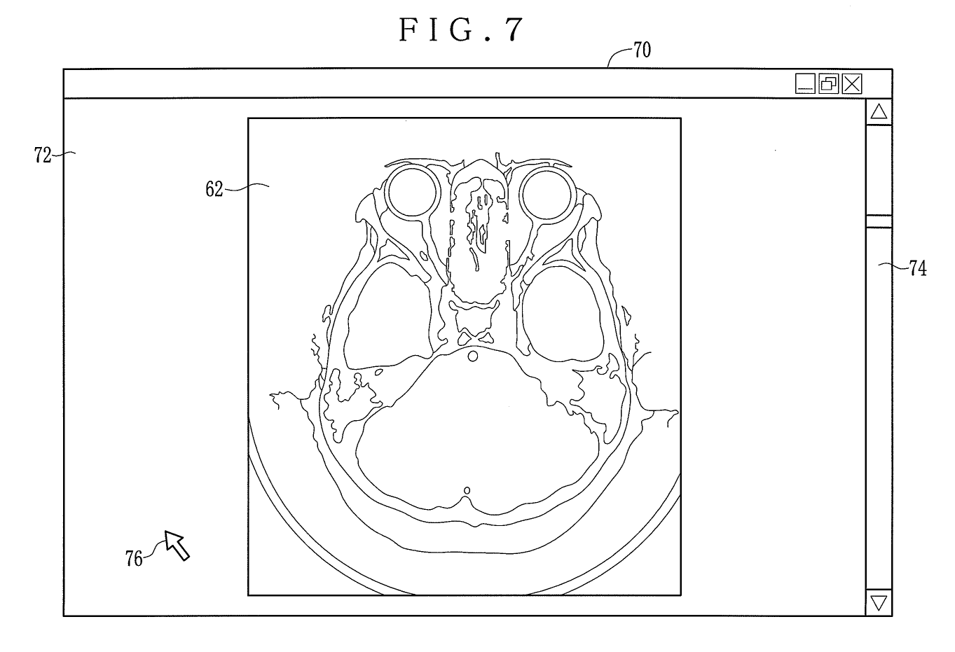

FIG. 7 shows an example of an image display screen 70 displayed on the image display monitor 43 of the image interpretation report making terminal 12. The image display screen 70 is displayed for example by selecting a particular request for an examination on a not-shown selection screen that shows a list of accepted examination requests. The image display screen 70 is provided with an image display area 72 for displaying the present image 62. On a right margin of the image display area 72 is displayed a scroll bar 74. A pointer 76 is also displayed on the image display screen 70, being movable on the image display screen 70 in corporation with the mouse 44.

As one of the accepted examination requests is selected on the list, the CPU 40 of the image interpretation report making terminal 12 accesses to the image database 55 of the image server 20, to read out the present data 60 that correspond to the selected examination request. Then, the CPU 40 constitutes the image display screen 70 such that the respective present images 62 contained in the read present data 60 are arranged in the image display area 72 in the sequence of their imaging time, and displays the image display screen 70 on the image display monitor 43.

Moreover, the CPU 40 displays the present image 62 in an approximately maximum size relative to the image display screen 70, as shown in FIG. 7. The present image 62 displayed on the image display screen 70 is switchable by scrolling the image display area 72. The image display area 72 may be scrolled for example by rotating a wheel of the mouse 44 or drugging the scroll bar 74. The method of displaying the present image 62 is not limited to the illustrated embodiment, but it is possible to display many present images 62 in an array on the image display screen 70.

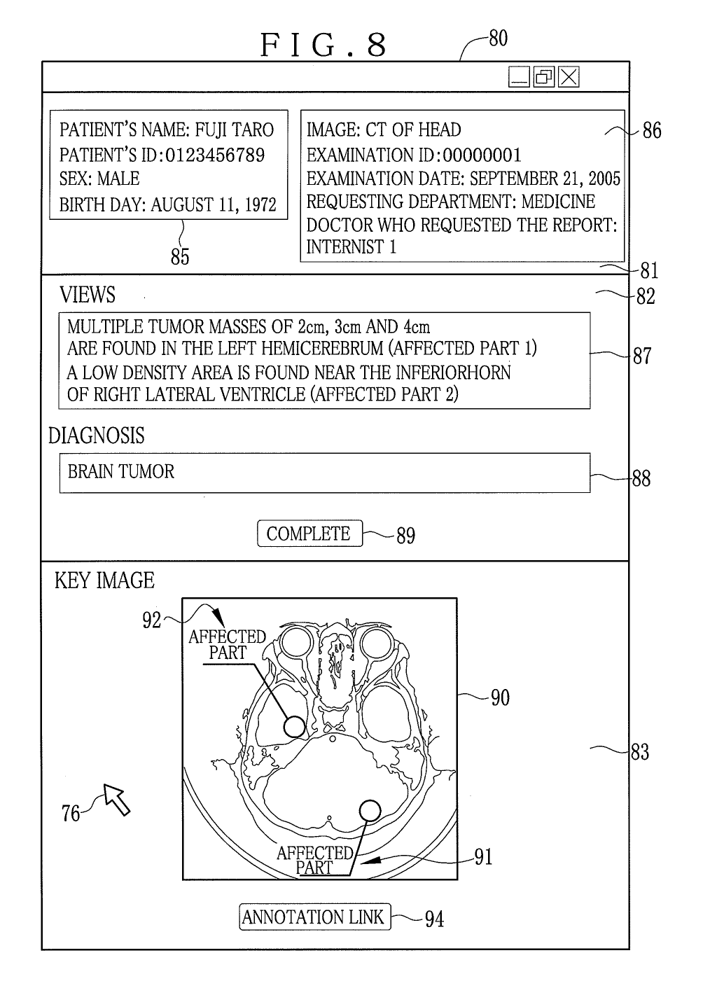

FIG. 8 shows an example of a report creation screen 80 displayed on the main monitor 42. Like the image display screen 70 on the image display monitor 43, the report creation screen 80 is displayed on the main monitor 42 when a particular request for an examination is selected. On the report creation screen 80, the pointer 76 is displayed as a common device to the image display screen 70. Where the image interpretation report making terminal 12 has the dual monitor structure, the common pointer 76 goes back and forth between the image display screen 70 and the report creation screen 80 according to the amount of operation on the mouse 44.

The report creation screen 80 is provided with an information display zone 81 for displaying various information, a comment input zone 82 for inputting comments such as findings, opinions and diagnoses, and a key image pasting zone 83 for pasting a key image 90. The information display zone 81 is provided with a patient data display box 85 and an examination data display box 86. The boxes 85 and 86 respectively display patient information and examination information, which are related to the selected examination request.

The comment input zone 82 is provided with a text input box 87 for inputting at least a finding as a comment, the finding explaining a condition seen from the image, and a second text input box 88 for inputting a diagnosis as a comment. The comment input zone 82 is also provided with a complete button 89 for terminating creation of the image interpretation report 24. The diagnosis describes a disease and an impression, which are derived from the finding. Note that the comments written in the image interpretation report 24 are not limited to the findings and diagnoses, but may include a suggestion for a countermeasure and other notes.

The doctor in charge of the image interpretation observes and interprets the respective present images 62 on the image display screen 70, and inputs the finding and the diagnosis in the respective text input boxes 87 and 88 through the keyboard 45, to write the image interpretation report 24. To complete making the image interpretation report 24, the doctor clicks the mouse 44 on the complete button 89. When the complete button 89 is clicked on, the CPU 40 of the image interpretation report making terminal 12 stores the image interpretation report 24 in the report server 22. At the same time, the CPU 40 informs of the completion of the image interpretation, for example, by e-mailing to the doctor who requested for the image interpretation report 24.

The key image 90 pasted in the key image pasting zone 83 is such an image that attracted attention of the doctor in charge of the image interpretation during the image interpretation. The key image 90 is produced by subjecting one of the present images 62 to image processing such as magnification or reduction, gradation control and the like. For example, the key image 90 is produced by drug-and-dropping one of the present images 62 from the image display screen 70 into the key image pasting zone 83.

On the key image 90, annotations 91 and 92 are overlaid or superimposed, each of which consists of a mark locating a concerned site where the doctor in charge of the image interpretation finds an affected part or an illness. The annotation 91 or 92 is superimposed on the key image 90, for example, by indicating the concerned site with the pointer 76 and then inputting text data through the keyboard 45. The annotations 91 and 92 as well as the key image 90 are stored as parts of the image interpretation report 24 in the report server 22. Although the example shown in FIG. 8 shows only one key image 90 and two annotations 91 and 92, how many key images and annotations are to display depends on the number of affected parts found in the images 62.

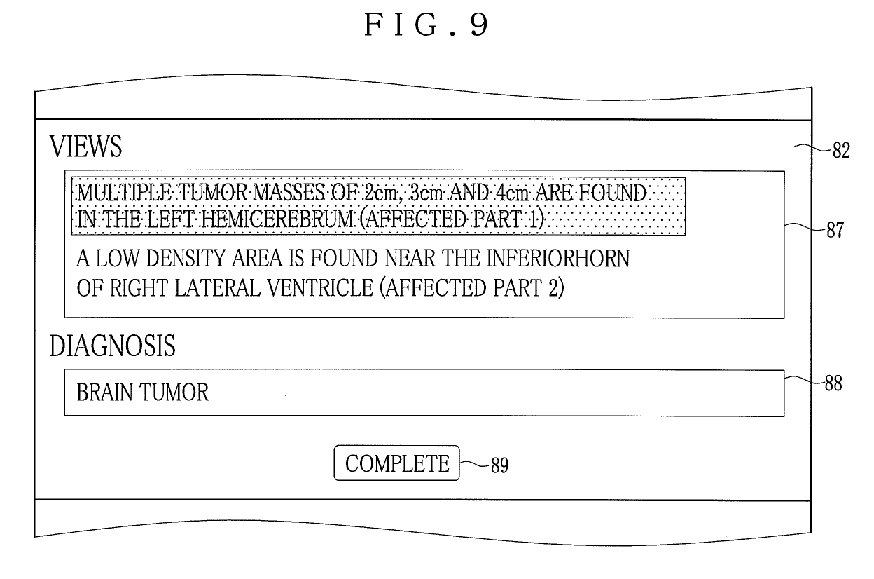

The key image pasting zone 83 is also provided with an annotation link button 94, which is used for linking the individual annotation 91 or 92 to the finding or diagnosis as inputted in the comment input zone 82. When the annotation link button 94 is clicked, the CPU 40 of the image interpretation report making terminal 12 starts executing a linkage process for linking either of the annotations 91 and 92 to the finding or diagnosis. For example, a certain word or phrase in the finding or the diagnosis as written in the text input box 87 or 88 is selected by drugging from the text input box 87 or 88 and, thereafter, a corresponding one of the annotations is clicked on, thereby linking the finding or diagnosis to the corresponding annotation. As shown for example in FIG. 9, when a finding concerning the affected part #1, which is written in the text input box 87, is selected by drugging, the selected finding is highlighted. Thereafter when the annotation 91 corresponding to the affected part #1 is clicked, the finding concerning the affected part #1 is linked to the annotation 91.

After making the linkage of the respective findings and diagnoses to the corresponding annotations, the CPU 40 creates annotation link data that represent linkage of the respective findings and diagnosis to the corresponding annotations. The annotation link data consists, for example, of data of a text start position and a text end position of the individual finding or diagnosis, and identification data of each annotation. The created annotation link data is recorded as metadata, for example, in the image interpretation report 24.

Since the findings and diagnosis are linked to the corresponding annotations, it comes to be possible to display a particular finding or diagnosis and the corresponding annotation automatically in an enhanced or discriminated manner as the doctor who requested for and is referring to the image interpretation report 24 designates the particular finding or diagnosis or the corresponding annotation, for example, by putting a pointer on the text of the particular finding or diagnosis or on the corresponding annotation. Enhancing the association between the designated finding or diagnosis and the corresponding annotation on the display screen clarifies the relation between the finding or diagnosis and the location of the related affected part in the image. Thus, the visual facility of the image interpretation report 24 is improved.

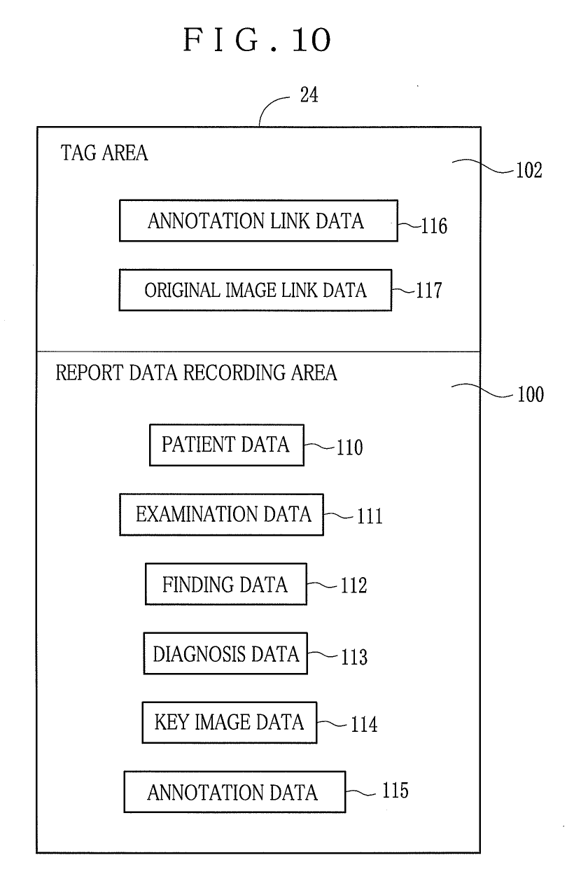

FIG. 10 schematically shows a structure of the image interpretation report 24 as created on the report creation screen 80. The image interpretation report 24 has a report data recording area 100 for recording various data representative of the contents of the image interpretation report 24, and a tag area 102 for recording metadata. For example, patient data 110 as displayed in the patient data display box 85, examination data 111 as displayed in the examination data display box 86, finding data 112 expressing a letter string of the finding written in the text input box 87, diagnosis data 113 expressing a letter string of the diagnosis written in the text input box 88, key image data 114 expressing the key image 90 as pasted on the key image pasting zone 83, and annotation data 115 expressing the respective annotations 91 and 92 as overlaid on the key image 90 are recorded in the report data recording area 100.

The key image data 114 may directly represent image data of the key image 90 or indicate memory addresses of the key image 90. For example, the annotation information 115 consists of identification data for identifying the individual annotations 91 and 92, coordinate data representative of respective locations of the annotations 91 and 92 and input text data.

In the tag area 102, on the other hand, for example, annotation link data 116 and original image link data 117 are recorded. The annotation link data 116 is created by making a linkage of a finding or diagnosis to an annotation, and is recorded in the tag area 102. The original image link data 117 is for linking the key image 90 to an original medical image. Concretely, the original image link data 117 represents a memory address of the original medical image as an origin of the key image 90. The original image link data 117 is recorded in the tag area 102 when the key image 90 is created, for example, by drug-and-dropping the original image into the key image pasting zone 83. Since the key image 90 is liked to the original medical image as the origin of the key image 90 via the original image link data 117, the doctor who requested for and is referring to the image interpretation report 24 can retrieve the original medical image automatically by clicking on the key image 90, without making a search procedure.

FIG. 11 shows an example of a mode selection screen 120 that is displayed on the main monitor 42 in advance to the report creation screen 80, when a request for examination is selected on a request selection screen or the like. The image interpretation report making terminal 12 has a new report making mode and a past report utilizing mode, and the mode selection screen 120 is provided with a new report making mode selection button 121 and a past report utilizing mode selection button 122. The new report making mode is for making the image interpretation report 24 from scratch, whereas the past report utilizing mode is for making the image interpretation report 24 on the basis of a past image interpretation report.

When the new report making mode selection button 121 is clicked to select the new report making mode, the CPU 40 of the image interpretation report making terminal 12 causes the main monitor 42 to display the report creation screen 80 with the text input boxes 87 and 88 as well as the key image pasting zone 83 blanked. Then, the doctor writes the findings and diagnosis in the text input boxes 87 and 88, pastes the key image 90 in the key image pasting zone 83, displays the annotations 91 and 92, and makes the linkage of the findings and diagnosis to the respective annotations 91 and 92, thereby making the image interpretation report 24.

On the other hand, when the past report utilizing mode selection button 122 is clicked to select the past report utilizing mode, the CPU 40 makes an access to the information management server 18, to judge whether the selected request for examination relates to an initial examination of a patient, or an examination for follow-up observation of the patient, by checking if the patient has ever gone through an examination under the same conditions as the selected request for examination.

If the CPU 40 judges that the selected request for examination relates to the initial examination, the CPU 40 displays a warning window 124 on the mode selection screen 120, to warn that there are not any past image interpretation report that can be copied to utilize for making a new report. Thereafter when an OK button 125 in the warning window 124 is clicked, the CPU 40 starts making the image interpretation report 24 in the new report making mode. Instead of the warning window 124, the warning may be done by use of a sound or a specific lamp or the like. It is alternatively possible to make the judgment as to whether the selected request for examination relates to a follow-up examination or not in advance to displaying the mode selection screen 120, so as to invalidate or not to display the past report utilizing mode selection button 122 when it is judged that the selected request for examination relates to an initial examination.

On the other hand, if the CPU 40 judges that the selected request for examination relates to a follow-up examination, the CPU 40 makes an access to the report server 22 to read out a previous image interpretation report, which was created concerning the previous examination before the selected request for examination. Hereinafter, the previous image interpretation report will be referred to as a past report 130, whereas an image interpretation report to be created on the basis of the past report 130 will be referred to as a present report 132 (see FIG. 13).

After reading out the past report 130, the CPU 40 copies the finding data 112, the diagnosis data 113, the annotation data 115 and the annotation link data 116 of the past report 130 in the present report 132.

After copying the respective data 112, 113, 115 and 116 of the past report 130 in the present report 132, the CPU 40 refers to the original image link data 117 of the past report 130, to determine the past image 66 that was used for the key image 90 of the past report 130. Thereafter, the CPU 40 makes an access to the image server 20 to read out the correspondence data 58 relating to the determined past image 66 from the correspondence data database 56. With reference to the read correspondence data 58, the CPU 40 determines which present image 62 corresponds to the key image 90 of the past report 130 with respect to their imaging positions.

After determining the present image 62 that corresponds to the key image 90 of the past report 130, the CPU 40 reads out the determined present image 62 as an original image for a present key image from the image database 55 of the image server 20. Then, the CPU 40 constitutes the original image link data 117 corresponding to the present image 62 as read out from the image database 55, and records the original image link data 117 in the tag area 102 of the present report 132. Simultaneously, the CPU 40 creates a key image 90 of the present report 132 from the read present image 62, constitutes the key image data 114 corresponding to the present key image 90, and records the key image data 114 in the report data recording area 100 of the present report 132.

As described so far, when the past report utilizing mode is selected, the CPU 40 records various data relating to the past images 66, as contained in the past report 130, in the present report 132. On the basis of the present report 132 having the data of the past report 130 recorded therein, the CPU 40 produces the report creation screen 80 and displays it as a template on the main monitor 42. Note that the patient data 110 and the examination data 111 are recorded in the report data recording area 100 of the present report 132 by reading out necessary information from the information management server 18, for example, on the basis of the examination ID or other data contained in the request for examination.

Thus, when the past report utilizing mode is selected, the report creation screen 80 is displayed with its text input boxes 87 and 88 filled with those findings and diagnosis which have been written in the past report 130. Beside that, in the past report utilizing mode, the key image 90 is displayed in the key image pasting zone 83 of the report creation screen 80, the key image 90 being newly created from one of the present images 62 that corresponds to the key image 90 of the past report 130 with respect to their imaging positions. Also an annotation is displayed at the same position as in the past report 130. Therefore, in the past report utilizing mode, the doctor can instantly create the present report 132 relating to the follow-up examination just by rewriting some parts of the copied findings and diagnosis.

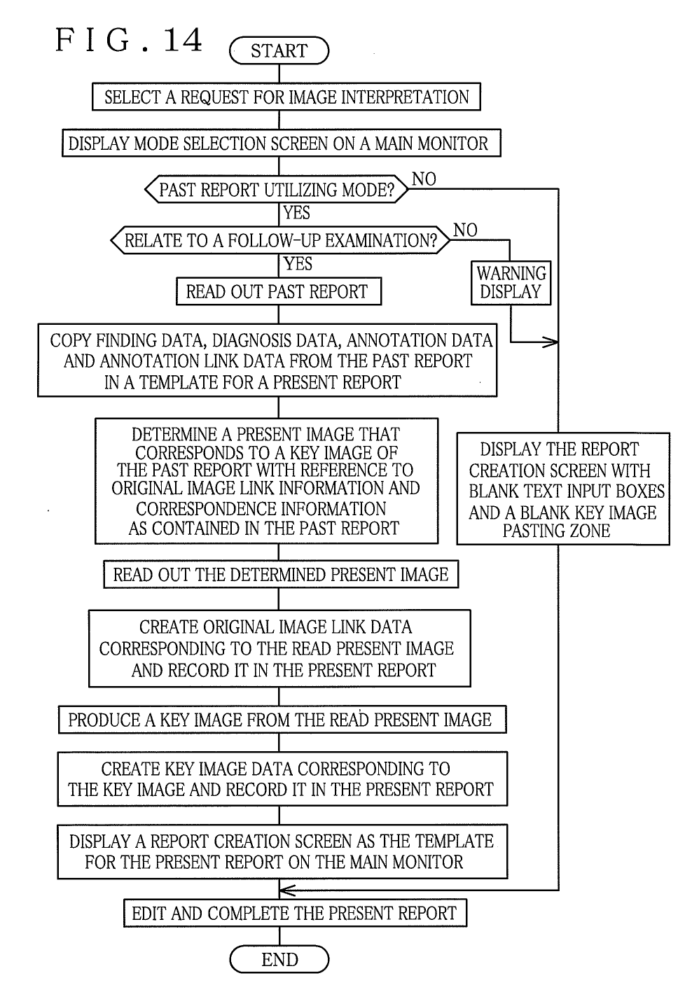

Now the operation of the medical network system 2 having the above configuration will be described with reference to the flowchart shown in FIG. 14.

A doctor in charge of the image interpretation in the inspection department interprets medical images and makes the present report 132 on the image interpretation report making terminal 12. The doctor selects a particular request for examination on the not-shown selection screen of the main monitor 42 of the image interpretation report making terminal 12, to start the image interpretation.

When the particular request for examination is selected, the mode selection screen 120 is displayed on the main monitor 42, as is shown in FIG. 11. The doctor clicks on either the new report making mode selection button 121 or the past report utilizing mode selection button 122, to choose between the new report making mode and the past report utilizing mode, for making the present report 132 relating to the selected request for examination.

When the new report making mode selection button 121 is clicked on to choose the new report making mode, the main monitor 42 displays the report creation screen 80 with its text boxes 87 and 88 as well as the key image pasting zone 83 blanked. Simultaneously, the present data 60 corresponding to the selected request for examination is read out from the image database 55, and the present images 62 contained in the read present data 60 are displayed one by one on the image display screen 70 of the image display monitor 43 (see FIG. 7). While interpreting the present images 62 on the image display screen 70, the doctor writes some findings and diagnosis, pastes the key image 90, displays the annotations 91 and 92, and links the findings and diagnosis to the respective annotations 91 and 92 on the report creation screen 80 of the main monitor 42, to make an image interpretation report 24 as the present report 132.

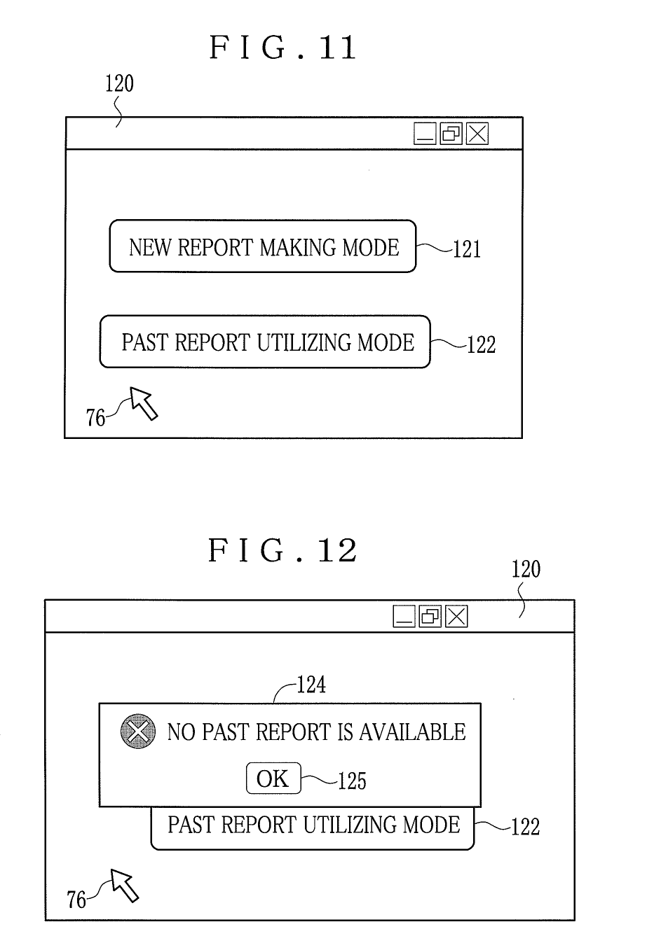

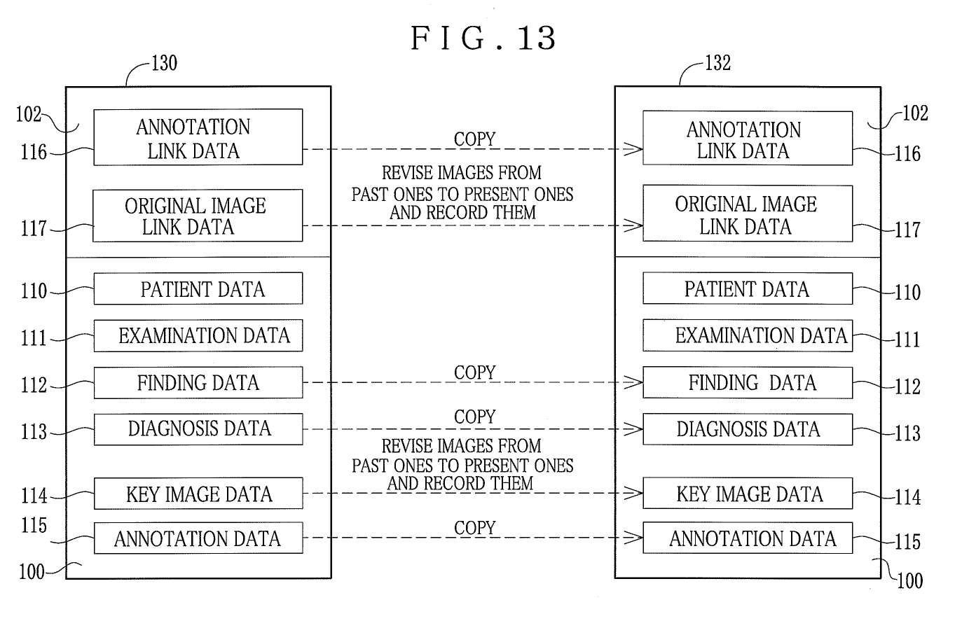

On the other hand, when the past report utilizing mode selection button 122 is clicked to select the past report utilizing mode, the judgment as to whether the selected request for examination relates to an initial examination of a patient, or an examination for follow-up observation of the patient. If it is judged that the selected request for examination relates to the initial examination, the warning window 124 is displayed over the mode selection screen 120, as shown in FIG. 12, to warn that no past report 130 is available for copy to utilize in the present report 132. When the doctor clicks on the OK button 125 in the warning window 124, the terminal 12 is reset to the new report making mode, and the report creation screen 80 having the blank text boxes 87 and 88 and the blank key image pasting zone 83 is displayed on the main monitor 42.

On the contrary, if the selected request for examination is judged to relate to the follow-up examination, a past report 130 related to the selected request for examination is read out from the report server 22, and the finding data 112, the diagnosis data 113, the annotation data 115 and the annotation link data 116 of the past report 130 are copied in the present report 132.

After copying the data 112, 113, 115 and 116 in the present report 132, the correspondence data 58 is referred to on the basis of the original image link data 117 that is written in the past report 130, to determine a present image 62 that corresponds to a past image 66 with respect to their imaging positions, wherein the past image 66 being an origin of a key image 90 of the past report 130. Then, the determined present image 62 is read out from the image database 55.

As the present image 62 is read out, original image link data 117 is newly created corresponding to the read present image 62, and is written in the tag area 102 of the present report 132. As the original image link data 117 is written in the present report 132, a key image 90 is created from the read present image 62, and key image data 114 corresponding to the created key image 90 is produced and written in the report data recording area 100 of the present report 132. Note that the sequence of procedures for creating and recording the respective data is not limited to the above embodiment.

After the respective data 112 to 117 are recorded in the present report 132, a report creation screen 80 is produced on the basis of this present report 132, and is displayed as a template for making the present report 132 on the main monitor 42. Thus, in the past report utilizing mode, the report creation screen 80 is displayed with its text input boxes 87 and 88 filled with those findings and diagnosis which have been written in the past report 130. Moreover, in the past report utilizing mode, the key image 90 is automatically created from one of the present images 62 that corresponds in imaging position to the key image 90 of the past report 130, and is displayed in the key image pasting zone 83 of the report creation screen 80. Also annotations are displayed at the same positions as in the past report 130. Consequently, in the past report utilizing mode, the doctor can instantly create the present report 132 just by rewriting some parts of the copied findings and diagnosis, without the need for making such operations as searching for the present image that corresponds the original past image of the past key image, and pasting it as the key image of the present report.

Although the slice images 30 taken by the CT scanner 16 are referred to as the present and past images 62 and 66 in the above-described embodiment, the present and past images 62 and 66 are not limited to this embodiment, but may be those taken by a MRI scanner or PET scanner. Moreover, in the above-described embodiment, an axial or transaxial slice image, which is taken in a perpendicular direction to a body axis of the patient, is shown as an example of the slice image 30, but the slice image 30 may be a sagittal slice image or a coronal slice image. Furthermore, the present and past images 62 and 66 are not limited to the slice images or tomograms, but may for example be transparent images taken by a CR machine.

Although the above embodiment has been described with respect to a case where there is only one past report available for producing a template of the present report, there may be cases where a plural number of examinations have been carried out before the present examination. In that case, the doctor may select one from among past reports on these past examinations, to copy it for producing the template of the present report.

Although the correspondence data 58 is produced in the image server 20 in the above embodiment, the correspondence data 58 may be produced in another device, for example, in the department terminal 10 or in the image interpretation report making terminal 12.

Although the present invention has been described with reference to the medical network system 2 where the device under test is a patient, the present invention is not to be limited to the above embodiment, but may be applicable to an examination system where the device under test is an animal, or a nondestructive inspection system where the device under test is an object or article.

Although the medical network system of the above embodiment is an intra network system for use in a single medical facility, the present invention is not limited to this embodiment, but may be applicable to a network system networking a plurality of medical facilities. Moreover, the present invention may be configured as a software program.

Thus, the present invention is not to be limited to the above embodiments but, on the contrary, various modifications will be possible without departing from the scope of claims appended hereto.

What is claimed is:

1. An image interpretation report making system comprising:
a data storage device for storing past data obtained through a past examination done on a test device, and present data lately obtained through an examination of said test device under similar condition to said past examination, wherein said past data includes past images taken from said test device at diverse imaging positions, whereas said present data include present images taken from said test device at similar imaging positions to said past images;
a report storage device for storing a past report depicting an interpretation of said past images, and a present report depicting an interpretation of said present images;
a correspondence data producing device for producing correspondence data that shows correspondence between said present images and said past images with respect to their imaging positions; and
a template producing device for producing a template for said present report on the basis of said correspondence data and said past report.

2. An image interpretation report making system as recited in claim 1, further comprising a display device for displaying said template and an operation device for editing said template as displayed on said display device, to complete said present report.

3. An image interpretation report making system as recited in claim 1, wherein said template producing device refers to said correspondence data to produce information relating to said present images from information relating to said past images that is included in said past data, and produces said template on the basis of said information relating to said present images.

4. An image interpretation report making system as recited in claim 3, wherein said past report includes a key image as selected from said past images, and said information relating to said past images includes past key image data that shows said key image of said past report, and wherein said template producing device produces a present key image and present key image data from one of said present images that is retrieved from said data storage device with reference to said correspondence data and said past key image data, and includes said present key image data in said information relating to said present images.

5. An image interpretation report making system as recited in claim 4, wherein said information relating to said past images includes annotation data that represents an annotation consisting of text data and a mark that is put on said past key image, and said template producing device copies said annotation data and includes it as annotation data for said present key image in said information relating to said present images.

6. An image interpretation report making system as recited in claim 5, wherein said information relating to said past images includes link data that links said annotation to comments written in said past report, and said template producing device copies said link data to include it in said information relating to said present images.

7. An image interpretation report making system as recited in claim 6, wherein said comments include a finding that describes a condition seen from said past images and/or a diagnosis that describes a symptom or an impression derived from said finding.

8. An image interpretation report making system as recited in claim 1, wherein said template producing device copies comment data in said template, said comment data representative of comments written in said past report.

9. An image interpretation report making system as recited in claim 8, wherein said comments include at least a finding that describes a condition seen from said past images and/or a diagnosis that describes a symptom or an impression derived from said finding.

10. An image interpretation report making apparatus for making a present report on the basis of a past report, wherein said past report depicts an interpretation of past images taken from a test device at diverse imaging positions at a past examination, whereas said present report depicts an interpretation of present images lately taken from said test device under similar condition to said past examination, said image interpretation report making apparatus comprising:
a correspondence data producing device for producing correspondence data that shows correspondence between said present images and said past images with respect to their imaging positions; and
a template producing device for producing a template for said present report on the basis of said correspondence data and said past report.

11. An image interpretation report making apparatus as recited in claim 10, further comprising a display device for displaying said template and an operation device for editing said template as displayed on said display device, to complete said present report.

12. An image interpretation report making method for making a present report on the basis of a past report, wherein said past report depicts an interpretation of past images taken from a test device at diverse imaging positions at a past examination, whereas said present report depicts an interpretation of present images lately taken from said test device under similar condition to said past examination, said image interpretation report making method comprising steps of:
producing correspondence data that shows correspondence between said present images and said past images with respect to their imaging positions; and
producing a template for said present report on the basis of said correspondence data and said past report.
